SEQUENCE LISTING (31 sequences)

SEQ ID NO: 1 - protein, 338 aa, Homo sapiens
MINSTSTQPPDESCSQNLLITQQIIPVLYCMVFIAGILLNGVSGWIFFYVPSSKSFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 2 - DNA, 1017 bp, Homo sapiens
atgatcaattcaacctccacacagcctccagatgaatcctgctctcagaacctcctgatcactcagcagatcattcctgtgctgtactgtatggtcttcattgcaggaatcctactcaatggagtgtcaggatggatattcttttacgtgcccagctctaagagtttcatcatctatctcaagaacattgttattgctgactttgtgatgagcctgacttttcctttcaagatccttggtgactcaggccttggtccctggcagctgaacgtgtttgtgtgcagggtctctgccgtgctcttctacgtcaacatgtacgtcagcattgtgttctttgggctcatcagctttgacagatattataaaattgtaaagcctctttggacttctttcatccagtcagtgagttacagcaaacttctgtcagtgatagtatggatgctcatgctcctccttgctgttccaaatattattctcaccaaccagagtgttagggaggttacacaaataaaatgtatagaactgaaaagtgaactgggacggaagtggcacaaagcatcaaactacatcttcgtggccatcttctggattgtgtttcttttgttaatcgttttctatactgctatcacaaagaaaatctttaagtcccaccttaagtcaagtcggaattccacttcggtcaaaaagaaatctagccgcaacatattcagcatcgtgtttgtgttttttgtctgttttgtaccttaccatattgccagaatcccctacacaaagagtcagaccgaagctcattacagctgccagtcaaaagaaatcttgcggtatatgaaagaattcactctgctactatctgctgcaaatgtatgcttggaccctattatttatttctttctatgccagccgtttagggaaatcttatgtaagaaattgcacattccattaaaagctcagaatgacctagacatttccagaatcaaaagaggaaatacaacacttgaaagcacagatactttgtga

SEQ ID NO: 3 - protein, 4 aa, Homo sapiens
HAVR

SEQ ID NO: 4 - protein, 4 aa, Homo sapiens
HALR

SEQ ID NO: 5 - protein, 4 aa, Homo sapiens
HATR

SEQ ID NO: 6 - protein, 4 aa, Homo sapiens
HATR

SEQ ID NO: 7 - protein, 4 aa, Homo sapiens
HATR

SEQ ID NO: 8 - protein, 4 aa, Homo sapiens
HVLR

SEQ ID NO: 9 - protein, 4 aa, Homo sapiens
HICR

SEQ ID NO: 10 - protein, 4 aa, Homo sapiens
HTLR

SEQ ID NO: 11 - protein, 4 aa, Homo sapiens
HVIR

SEQ ID NO: 12 - protein, 4 aa, Homo sapiens
HTVR

SEQ ID NO: 13 - protein, 4 aa, Homo sapiens
HLTR

SEQ ID NO: 14 - protein, 4 aa, Homo sapiens
HLTR

SEQ ID NO: 15 - protein, 4 aa, Homo sapiens
HTVK

SEQ ID NO: 16 - DNA, 12 bp, Homo sapiens
cacgcggtgaag

SEQ ID NO: 17 - DNA, 12 bp, Homo sapiens
cacgcattgcgg

SEQ ID NO: 18 - DNA, 12 bp, Homo sapiens
cacgcgacaaga

SEQ ID NO: 19 - DNA, 12 bp, Homo sapiens
catgcgacccgg

SEQ ID NO: 20 - DNA, 12 bp, Homo sapiens
catgccactaga

SEQ ID NO: 21 - DNA, 12 bp, Homo sapiens
cacgcgttgcgt

SEQ ID NO: 22 - DNA, 12 bp, Homo sapiens
catatttgccgg

SEQ ID NO: 23 - DNA, 12 bp, Homo sapiens
cacacgctgcga

SEQ ID NO: 24 - DNA, 12 bp, Homo sapiens
cacgttatccga

SEQ ID NO: 25 - DNA, 12 bp, Homo sapiens
catgtgacaagg

SEQ ID NO: 26 - DNA, 12 bp, Homo sapiens
cacttgacgcgt

SEQ ID NO: 27 - DNA, 12 bp, Homo sapiens
catttaacaagg

SEQ ID NO: 28 - DNA, 12 bp, Homo sapiens
cataccgtcaag

SEQ ID NO: 29 - protein, 4 aa, Homo sapiens
HIAR

SEQ ID NO: 30 - protein, 5 aa, Artificial Sequence
  Synthetic
AVILT

SEQ ID NO: 31 - protein, 5 aa, Artificial Sequence
  Synthetic
VILTC

(19) United States
(12) Patent Application Publication
(10) Pub. No.: US 2007/0154947 A1
(43) Pub. Date: Jul. 5, 2007

(54) Chemical biodiscriminator

(75) Inventors: James R. Broach, Skillman, NJ (US); Addison D. Ault, Princeton, NJ (US)

Correspondence Address: MEDLEN & CARROLL, LLP, Suite 350, 101 Howard Street, San Francisco, CA, 94105, US

(73) Assignee: The Trustees of Princeton University

(21) Appl. No.: 11/513,594

(22) Filed: Aug. 31, 2006

Related U.S. Application Data
(60) Provisional application No. 60/712,799, filed on Aug. 31, 2005.
(60) Provisional application No. 60/801,898, filed on May 19, 2006.

(51) Int. Cl. G01N 33/53; G01N 33/569; C07K 16/28

(52) U.S. Cl. 435/7.1; 435/7.31; 530/388.22

(57) ABSTRACT

In one embodiment the invention provides a mutant UDP-glucose receptor (P2Y14) functionally expressed in the yeast Saccharomyces. The mutant receptors have ligand-binding properties that are useful as practical biosensors. Mutagenesis of the entire UDP-glucose receptor gene yielded receptors with increased activity but similar ligand specificities, while random mutagenesis of residues in the immediate vicinity of the ligand-binding pocket yielded mutants with altered ligand specificity. The receptor mutants can be used to detect chemical ligands in complex mixtures and to discriminate among chemically or stereochemically related compounds. Also provided are methods for combinatorial applications wherein engineered receptors can be applied, for example, in a pairwise manner to differentiate among several chemical analytes that would be indistinguishable with a single receptor.

### CROSS-REFERENCE TO RELATED APPLICATION

This application claims the benefit of provisional U.S. Application No. 60/712,799, filed Aug. 31, 2005, and provisional U.S. Application No. 60/801,898, filed May 19, 2006, both incorporated herein in their entirety by reference for all purposes.

### GOVERNMENTAL SUPPORT

This work was supported in part by National Institutes of Health Grants GM 48540, CA 41086 and F32 DC 0055580. Consequently, the United States Government may have certain rights to this invention.

### FIELD

The present invention relates to chemical sensors. More particularly, the invention relates to biosensors, especially to biosensors capable of discriminating among closely related chemical species, and to methods of making and selecting sensor elements for use in a biodiscriminator.

### BACKGROUND

Some receptors mediate chemical communications between cells. Others, especially G protein coupled receptors (“GPCRs”) function as “input ports” in sensor systems that enable an organism to perceive its environment. Receptors involved in intercellular communication are often exquisitely adapted or “tuned” to respond to a particular chemical, thus recognizing only that particular chemical as a signal, while excluding others. For example, a particular receptor may respond to serotonin, but not to structurally similar tryptophan or melatonin.

In contrast, a receptor primarily responsible for capturing environmental signals normally responds to a broad range of stimuli. Nature, apparently as an alternative to the infeasible mechanism of providing a specific receptor for each conceivable stimulus, utilizes a few broad-spectrum receptors to discriminate among many stimuli. For example, the human visual system distinguishes among a huge diversity of colors using only three different receptors. The mammalian olfactory system can distinguish among thousands of compounds using only 350-1200 receptors.

In the olfactory system, a single compound can bind to and activate a number of different receptors, and each receptor can respond in varying degrees to a number of related compounds. It is vital that any ensemble of such “promiscuous” receptors co-operate. The ensemble would be unlikely to “make sense” if the activation of each receptor in the ensemble and the downstream effects of activation had not been “selected” during evolution for breadth of coverage and if the ensemble were not governed by a combinatorial mechanism. A principal advantage of such systems, their extraordinary discriminatory power, resides in this combinatorial mechanism, a mechanism built with receptors that have overlapping specificities and that evolutionary pressure has selected over time.

### SUMMARY

The need for making sensor elements that can be used in biosensors according to the invention, several of which elements are provided herein, can be satisfied with the methods of the invention, as can the need for systematic selection of such elements for combinatorial recognition of analytes.

In one embodiment, the invention provides a plurality of sensor elements, wherein at least two of the sensor elements are capable of sensing a) independently, at least two chemical species, and b) in common, one of the at least two species. In a preferred embodiment, sensing comprises binding of at least one of the species. In another embodiment the sensor element comprises a biological molecule. In some embodiments, the biological molecule comprises a GPCR, which may be naturally occurring or a mutant made by random mutagenesis or site-directed mutagenesis. In one embodiment the GPCR is expressed in a living cell, where it may be coupled to a reporter system. In one embodiment, the living cell is a yeast cell. In a preferred embodiment, the mutant GPCR is hypersensitive as defined herein. In one embodiment, the plurality of sensor elements comprises a biodiscriminator as defined herein. In one embodiment, the chemical species sensed are isomers, which may be stereoisomers.

In another aspect, the invention provides a method of making a chemical sensor from sensor elements, the method comprising a) selecting a plurality of sensor elements of of the sensor wherein at least two of the sensor elements are capable of sensing (i) independently, at least two chemical species, and (ii) in common, one of those at least two species; and b) providing a common environment for sensing. In one embodiment, sensing comprises binding of a chemical species to a sensor element. In one important embodiment, a sensor element, sensitive to at least two chemical species, is unequally sensitive thereto. In some embodiments the sensor element is a biological molecule which, optionally, may be a GPCR, either naturally occurring or a mutant. In either case, the GPCR may be expressed in a living cell, preferably a yeast cell, preferably coupled to a reporter system. In some embodiments the mutant form is made by random mutagenesis. In some embodiments, the mutant is made by site-directed mutagenesis. In a preferred embodiment, at least one of the mutants is hypersensitive as that term is defined herein.

The method provides chemical sensors that are biosensors or biodiscriminators as defined herein. In some embodiments, such biosensors and biodiscriminators are capable of distinguishing among chemical species that are isomers, including stereoisomers.

In another aspect, the invention provides a method of optimizing an ensemble of chemical sensor elements comprising the steps of: a) assigning the sensor elements of said ensemble to similarity clusters and b) excluding from each said similarity cluster all but one of said sensor elements. Optionally, the ensemble may be tested for masking and metamerism according to some embodiments of the invention, and “tuned” by recursively applying the optimization method.

In another aspect, the invention provides specific GPCRs that may be used in biosensors and biodiscriminators of the invention. In one embodiment, the present invention contemplates a composition comprising an amino acid sequence that is at least 80%, and more preferably at least 90%, and still more preferably at least 95% identical to SEQ ID NO:01, wherein said amino acid sequence has amino acid substitutions at positions 54, 98, 193, 243, 251 and/or 252. It is not intended that the present invention be limited by the precise substitution. In one embodiment said substitution at position 54 consists of a glutamic acid. In one embodiment, said substitution at position 98 consists of a glycine. In another embodiment, said substitution at position 193 consists of a valine. In one embodiment, said substitution at position 243 consists of an isoleucine. In one embodiment, said substitution at position 251 consists of an alanine. In one embodiment, said substitution at position 252 consists of a valine. In one embodiment, all of the positions are substituted in the manner described above. In another embodiment, two positions (e.g. 54 and 98, or 251 and 252, etc.) are substituted in this manner.

The present invention also contemplates, in one embodiment, a composition comprising a first GPCR comprising an amino acid sequence at least 90% identical to SEQ ID NO:01, wherein said amino acid sequence comprises, at positions corresponding to positions 54, 98, 193 and 243, respectively, of SEQ ID:NO:01, a glutamic acid, a glycine, a valine and an isoleucine and, corresponding to position 252 of said SEQ ID NO:01, a V. In another embodiment, a second GPCR having the same amino acid sequence as the first GPCR is provided, except that the second GPCR has an alanine and a valine at positions 251 and 252, respectively. A third GPCR, in another embodiment, instead of the sequence of the first GPCR, has an alanine and a leucine, respectively, at positions 251 and 252. A fourth GPCR, in another embodiment, instead of the sequence of the first GPCR, has a valine and a leucine, respectively, at positions 251 and 252. A fifth GPCR, in another embodiment, at positions 251 and 252, respectively, has an isoleucine and a cysteine. A sixth GPCR, in another embodiment, instead of the sequence of the first GPCR, at positions 251 and 252, respectively, has a threonine and a leucine. A seventh GPCR, in another embodiment, instead of the sequence of the first GPCR, at positions 251 and 252, respectively, has a valine and an isoleucine. An eighth GPCR, in another embodiment, instead of the sequence of the first GPCR, at positions 251 and 252, respectively, has a valine and a threonine. A ninth GPCR, in another embodiment, instead of the sequence of the first GPCR, at positions 251 and 252, respectively, has a leucine and a threonine. A tenth GPCR, in another embodiment, instead of the sequence of the first GPCR, at positions 251 and 252, respectively, has an alanine and a threonine. An eleventh GPCR, in another embodiment, instead of the sequence of the first GPCR, at positions 251, 252 and 253, respectively, has a threonine, a valine and a lysine. Finally, a twelfth GPCR, in another embodiment, instead of the sequence of the first GPCR, at positions 251, 252 and 253 and 278, respectively, has a glutamic acid, a glycine, a valine and an isoleucine.

### DRAWINGS

The accompanying drawings and the description and appended claims that follow will provide further understanding of these and other features, aspects and advantages of the present invention.

FIG. 1 shows ligand response of wild type and mutant UDP-glucose receptors. Dose/response curves measured with three different ligands: UDP-glucose (circles), UDP-galactose (squares) and UDP (diamonds),

Panel A. Wild type (wt) UDPG receptor

Panel B. Mutant 2211

Panel C. Mutant H-20

Panel D. Mutant K-3

Note that wt and 2211 receptors have similar response patterns for the three ligands with different activation levels (note differences in scale of the two graphs) while H-20 and K-3 exhibit different relative ligand preferences.

FIG. 2 shows UDP antagonization of the activation of UDP-glucose receptor 2211.

Panel A. Inhibition of signaling by UDP. IC50 of UDP is 10^{-4.5} M.

Panel B. Schild regression of UDP antagonism of UDP-glucose agonist activity. Slope of the linear fit is ˜0.8, which is consistent with UDP having a weak partial agonist activity.

FIG. 3 Shows discrimination of chemical analytes using mutant receptors.

Panel A. Ratio of 2211:H-20 receptor activation over a range of concentrations from 10⁻⁴ to 10⁻⁵ M.

Panel B. Ratio of H-20:K-3 receptor activation over the same range of concentrations.

Panel C. Schematic representation of the rudimentary chemical sensors in which relative activation is indicated by a ‘+’ or ‘−’. Each pair of the indicated measurements can uniquely identify one of the three ligands.

FIG. 4 depicts resolution of sensors with no crosstalk. Vertical axis is log scale ranging over four orders of magnitude. Red dot represents a data point while shaded area reprents uncertainty in the measurement projected onto the response gradient.

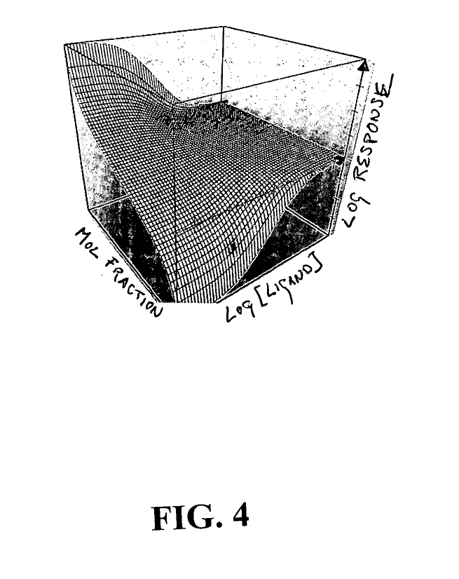

FIG. 5A is a graphic model based on UDP-glucose receptor mutants. The model assumes crosstalk between ligands and includes inhibitory terms. This pair of receptors will have lower resolution than the completely independent receptors.

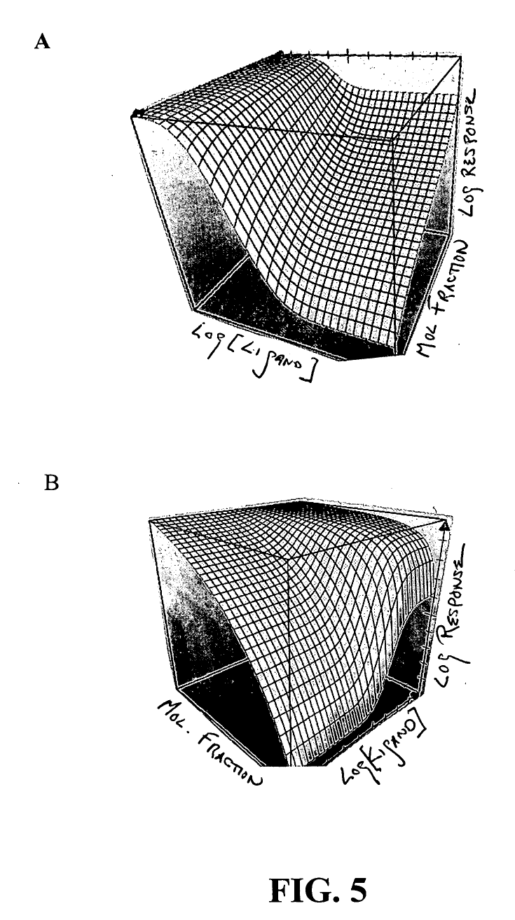

Figure FIG. 5B is a graphic model based on mutants 2211 and L-3 UDP-glc/UDP-glcNAc showing the relationship between full and partial agonists.

FIG. 6 shows the nucleotide sequence and the one-letter amino acid sequence for the human wild-type GPCR receptor for UDP-glucose.

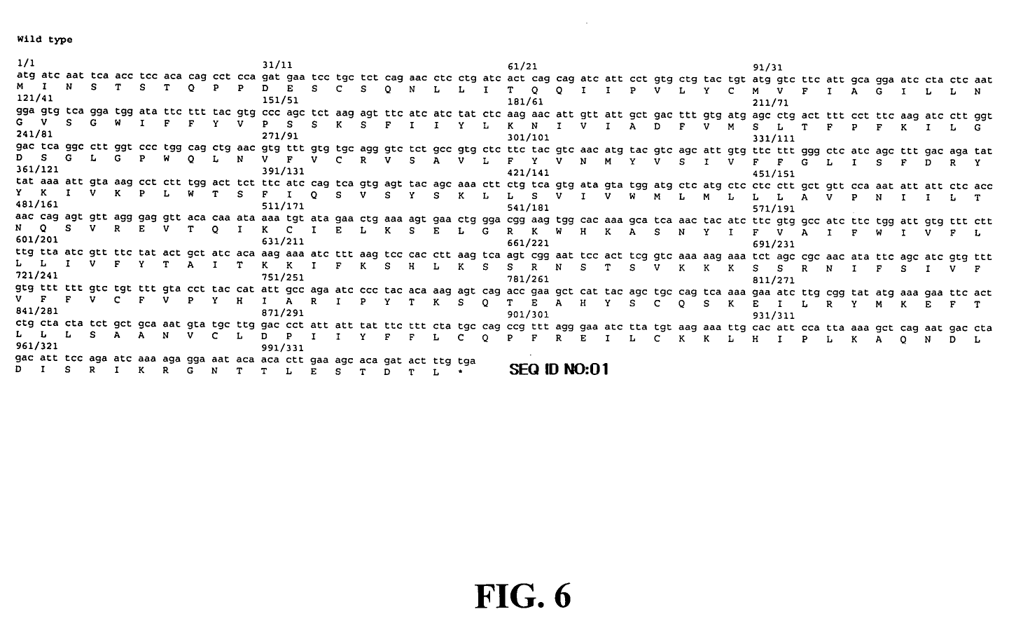

FIG. 7. shows a flow chart of steps for developing a biosensor of the invention.

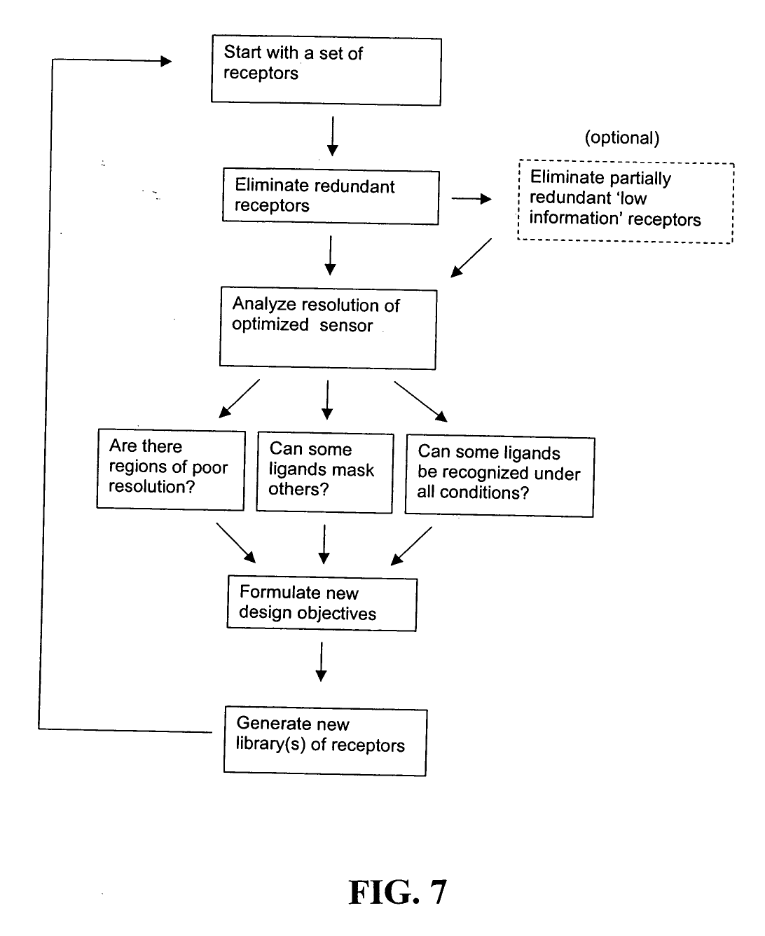

### DEFINITIONS

The term “hypersensitive” as used herein refers primarily to a mutant receptor that is more sensitive than its wild-type predecessor to representative members of the family of chemicals to which the wild-type is sensitive. Without wishing to be bound to any particular theory, it is thought that hypersensitivity obtains when the mutant binds a chemical more tightly (i.e., with higher affinity) than the wild-type receptor binds the same chemical. However, since binding may be quantified as the extent of a physiological or biochemical response of a biological system to which the receptor is coupled, such measures, normalized by the concentration of the chemical in the relevant solution or other environment, generally determine receptor sensitivity as a practical matter.

As used herein, an “ensemble” is a plurality of chemical sensing elements that co-operatively sense, detect, or quantify a chemical species or distinguish among a plurality of chemical species. An ensemble is distinguished from a mere catalogue, library, inventory or collection of sensing elements because each member of an ensemble, when functioning as a sensing element, shares a common environment. It is not intended, however, that this commonality must be achieved by dispersing every sensing element of an ensemble in a single solution or other homogeneous environment. By way of example and not limitation, each member may occupy a different test tube, a different well in a 96-well plate or a different spot in a microarray. The environment is said to be “common” as long as the results of the interactions between chemical species (or, interchangeably in this context, “analytes” or “ligands”) and sensor elements can be directly compared, within the limits conventionally achieved in the art of measuring discrete samples.

As used herein, an “n>m array” or “n>m ensemble” or “n>m sensor” is an array or ensemble of m sensing elements that is capable of distinguishing among n chemical species (or analytes or ligands), where n>m. Sensor elements in n>m array, may be any sensors, whether or not comprising biological molecules, as long as they are capable of interacting with chemical species with a measurable response. Accordingly, such n>m arrays are not limited to biosensors or biodiscriminators.

As used herein, a biosensor is any ensemble of biological sensing elements, including but not limited to an n>m ensemble, wherein a sensor element, upon interacting with an analyte, is capable of marking that interaction by reacting measurably. An ensemble is capable of detecting the presence of or assaying the amount of an analyte or chemical species (whether elemental, ionic, molecular, or supramolecular) in an environment. It is not intended that a biological sensing element or elements be limited to any particular level of biological organization. The element may, without limitation, be a biomolecule (e.g., a protein such as an enzyme or antibody, a nucleic acid such as an aptamer, or a polysaccharide such as an adhesin receptor), a virus or bacteriophage, a prokaryote or eukaryote, a portion of a cell or an extract thereof, a cellular organelle, a population of cells, an organism, or a population of organisms.

Analytes include but are not limited to chemical species. An analyte is any substance or material undergoing analysis. The relevant environment in which the analyte is dispersed may be a surface or a volume, which volume may contain a gas, liquid, solid or mixtures thereof. In the context of certain embodiments of the invention, a plurality of species may undergo analysis as a mixture. Thus, a test sample of the mixture may be referred to as the “analyte” in some contexts herein.

Biosensors may be referred to herein as “chemical detectors,” “sensor systems,” “chemical sensor systems,” “receptor arrays” or “chemosensory arrays.” Unless the context requires another meaning, these terms are used interchangeably.

As used herein, a biodiscriminator is a biosensor capable of detecting the presence of or assaying the amount of two or more substances in the same environment. Although a biodiscriminator comprises a plurality of detector elements, it is not intended that the concept of a biodiscriminator as used herein be limited to any particular configuration or assembly of detector elements. Each detector element of a biodiscriminator may reside in its own container (e.g., a test tube, a well of a 96-well-plate, a spot on a film) in an arrayed manner or otherwise. Alternatively, different elements may reside in the same solution, as long as the response of each element to an analyte is separately readable (e.g., by employing distinctive fluorescent tags having different absorption or emission spectra). Two or more analytes are said to be in the “same environment” or in a “common environment” either when they co-exist in one solution or other medium during analysis or when they are separately analyzed by the same biodiscriminator. In the latter case, two or more analytes may be analyzed simultaneously with a solitary biodiscriminator, or separately, as long as the biodiscriminators employed all have the same ensemble of detector elements.

As used herein, “resolving power” refers to the ability of any two sensor elements, paired by virtue of sharing a responsiveness to each of a given pair of ligands, to respond differently (to a predetermined degree of statistical confidence) to changes in the composition and concentration of a given mixture of analytes. Resolving power of a chemical sensor is thus analogous to the angular separation between two objects that is required to distinguish them as separate images in an optical device.

As used herein, GPCRs are G protein-coupled receptors, also known as 7-transmembrane (7TM) receptors. They reside in the membrane that envelops the living cell. When the extracellular region of a GPCR binds a ligand (which may be a small molecule, a peptide or a protein), the receptor's shape changes. The change disrupts an ongoing relationship between the intracellular region of the GPCR and certain intracellular molecules, typically a heterotrimeric G protein (other GPCR “partners” include proteins such as kinases, arresting, ligases, and even other GPCRs). Thus begins the propagation of a biochemical signal (i.e., “information”) into the cell from the cell's immediate environment. GPCRs may be naturally occurring receptors, as catalogued in online databases, or they may be engineered by altering the genes that encode them or by “tying” them to an engineered signaling system. They may also be engineered to send signals without being tied to a G protein or any intracellular biochemistry. A change in the shape of a suitably engineered GPCR may, for example, make the GPCR fluoresce.

As used herein, two GPCRs are said to have overlapping specificity when both are sensitive to a given ligand. They are commonly capable of sensing one of at least two ligands. One of the two GPCRs may be relatively more sensitive to the ligand than the other. The term also describes two GPCRs, each of which responds to (at least) a given pair of ligands, wherein one of the two GPCRs may have a preference for the first member of the ligand pair, and the other a preference for the second member of the ligand pair. A GPCR is said to be sensitive to a chemical if the chemical binds to the GPCR or if contact between the chemical and the GPCR results directly in a response mediated (typically) by a G protein. A GPCR is said to bind a chemical if the chemical activity of that chemical in solution with the GPCR is less than the chemical activity of the same physical mass of that chemical in the same solution without the GPCR.

“Sugar nucleotides” are nucleotides in which the nucleotide gamma phosphate is substituted with a sugar moiety. Examples of sugar nucleotides include the compounds UDP-glucose, ADP-glucose and dTDP-glucose, and the various isomers and derivatives of these molecules that can be derived from isomerizing or derivatizing the sugar moiety. For the compound UDP-glucose, such compounds would include UDP-galactose, UDP-glcNAc, UDP-galNAc, as well as numerous other sugar derivatives that are synthesized in the course of carbohydrate metabolism. Sugar nucleotides also include nucleotides linked to nonnatural sugar analogs and derivatives used by chemists for chemo-enzymatic synthesis of carbohydrates and carbohydrate analogs.

A “nucleic acid” is an organic substance in which hereditary information is stored and from which it can be transferred. Nucleic acid molecules are polymers comprising nucleotide monomeric units. The two chief types are DNA (deoxyribonucleic acid) and RNA. A nucleic acid “sequence” is a nucleic acid having nucleotide units disposed therein in a specific sequence.

As used herein, a motif is a recurring sequence of amino acids in a protein or a family or class of proteins, typically but not necessarily associated with a recurring secondary or tertiary structural feature of a protein such as a loop, fold or helix.

As used herein, “ligand space” or (or “chemical space”) refers to the complete ensemble of chemical ligands that the analyst would wish to analyze with a given chemical sensor or discriminator. The “ensemble” would comprise a range of chemical species, and different concentrations of each. For instance, if the sensor or discriminator is being deployed to screen a family of drug molecules, ligand space might encompass all analytes in the family at a plurality of physiologically relevant concentrations of each analyte.

As used herein, a ligand is a molecule that binds by intermolecular forces (usually non-covalent in nature) to a site on another molecule, typically a larger molecule or a macromolecule such as a GPCR, thereby changing the chemical conformation of the larger molecule.

As used herein, “preferentially sensitive” refers to a sensor element that is responsive to a plurality of ligands but responds (half-maximally, for example) to a lesser amount or concentration of one such ligand than any other such ligand. With respect to a sensor element for use in a biosensor or biodiscriminator, the requisite degree of difference is dependent upon the objectives for which the sensor is designed.

As used herein, a mutation is any chemical change in deoxyribonucleic acid (“DNA”) or ribonucleic acid (“RNA”) that changes the genetic code from what is encoded in naturally occurring or wild-type DNA or RNA. Such a change may manifest as a “point mutation,” that is, a change in a single element (“nucleotide”) in a DNA or RNA polymer (e.g., an exchange of a purine for a purine; an insertion or a deletion of a nucleotide), or as a “large-scale mutation,” that is, a change in a larger region of the polymer. Point mutations and large-scale mutations may lead to mutations in the proteins that the DNA or RNA encodes, as manifested by changes in the amino acid composition of the proteins. Such changes may alter the functions of the proteins, manifested as loss-of-function mutations, gain-of-function mutations, dominant negative mutations (wherein the mutated form of the protein antagonizes the wild-type protein) or lethal mutations (wherein the mutated form malfunctions sufficiently to prevent effective reproduction).

Some embodiments of the present invention provide secondary or tertiary mutant or variant forms of the mutant GPCRs described herein. It is possible to modify the structure of a peptide having an activity of the GPCRs described herein for such purposes as enhancing expression in a host cell, coupling efficiency, stability, and the like. For example, a modified peptide can be produced in which the amino acid sequence has been altered, such as by amino acid substitution, deletion, or addition. For example, it is contemplated that an isolated replacement of a leucine with an isoleucine or valine, an aspartate with a glutamate, a threonine with a serine, or a similar replacement of an amino acid with a structurally related amino acid (i.e., conservative mutations) will not have a major effect on the relevant biological activities of the resulting molecule (sensitivity and specificity for ligand, activation by ligand). Accordingly, some embodiments of the present invention provide variants of the mutant GPCRs described herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur -containing (cysteine and methionine) (See e.g., Stryer (ed.), Biochemistry, 2nd ed, WH Freeman and Co. [1981]). Whether a change in the amino acid sequence of a peptide results in a functional homolog can be readily determined by assessing the ability of the variant peptide to produce a response in a fashion similar to the wild-type protein using the assays described herein. Peptides in which more than one replacement has taken place can readily be tested in the same manner.

As used herein, a motif is a recurring sequence of amino acids in a protein or a family or class of proteins, typically but not necessarily associated with a recurring secondary or tertiary structural feature of a protein such as a loop, fold or helix.

As used herein, “combinatorial” refers to a strategy of identifying, detecting and/or quantifying an analyte by reading and analyzing, in combination, the responses of several sensor elements, each of which is responsive to the analyte to one degree or another.

“Random mutagenesis” refers herein to any method by which an amino acid sequence in a polypeptide is changed, whether at particular points in the sequence or by region, with the objective of producing a plurality of new sequence combinations, then checking (“screening”) each new combination for its effect(s) on a cell or other biological system, and finally, selecting from cells or organisms expressing the new sequences, specimens that exhibit the desired behavior (“phenotype”). A preferred method relies on first introducing changes in the nucleotide sequence of a nucleic acid polymer that encodes the polypeptide sought to be changed. Since nucleic acid polymers reproduce as “copies” under the control of a “polymerase” enzyme, choosing a polymerase enzyme that makes random copying errors (several error-prone polymerases are known in the art) provides an abundance of random mutations.

“Site-directed mutagenesis” refers herein to any method of introducing a change in the amino acid sequence of a polypeptide, at a particular, pre-determined position, typically by introducing into the underlying nucleic acid polymer a change in the polymer's nucleotide sequence such that the new sequence encodes the “new” polypeptide.

As used herein, a “similarity cluster” is any set of chemical sensor elements or receptors useful in a sensor system according to the invention, the set consisting of a plurality of elements wherein each element is identified by a value (which may, for example, be an eigenvalue) determined by the incremental change in resolution of a sensor system occasioned by deleting that element from the system, and wherein that value is, by a pre-determined criterion, “similar” to the corresponding value for all other members of the set. Similarity may be determined by any suitable statistical method of cluster analysis, such as methods described by Jain, A. K. and Murty, M. N., ACM Computing Surveys (1999) 31:264-323. Similarity clusters may be used to assist in the construction of the final array by selecting, for example, one sensor element from each cluster. Sets contributing below a predetermined threshold are deleted from consideration.

As used herein, “masking” refers to a condition that may be imposed upon a sensor system or discriminator by one of the analytes to which the system is exposed during use. For a non-limiting example, a system that highly resolves a first pair of analytes “A” and “B,” might poorly resolve a second pair of analytes “B” and “C” if C's affinities for receptors that are critical to the system's ability to distinguish A from B happen to be very much greater than A's or B's affinity therefor.

As used herein, “metamerism” refers to a condition that may occur in a region of ligand space, wherein different ligand combinations produce identical patterns of activation (as determined by the output signal) in the sensor system or discriminator that is being used to distinguish the ligands of that combination from one another at certain ligand concentrations.

A sensor element is said to be “redundant” in a sensing system according to the invention if its deletion from the system fails to change the output signal of the system and such failure cannot be attributed to masking or metamerism.

Description

Since the extraordinary discriminatory power of the olfactory system owes much to a combinatorial mechanism built with receptors that have overlapping specificities, it seemed useful to explore the concept of combinatorial recognition of analytes by GPCRs as a means of creating broad-specificity chemical detectors, especially ensembles of GPCRs that function collectively as chemical discriminators wherein m receptors are capable of distinguishing among n ligands, where n>m. Such systems would be more efficient than systems in which a ligand cannot be detected if the system happens not to include a specific receptor for that ligand. It would be useful, also, to explore the applicability of the concept to other receptor types, biological or otherwise.

One strategy to construct detectors or discriminators according to this “olfactory paradigm” is to exploit the naturally-occurring diversity of certain chemical receptors, including olfactory receptors. Olfactory receptors, however, are difficult to express outside of neuronal tissue, and naturally occurring non-olfactory receptors generally lack sufficient diversity for effective use in chemosensory arrays of n>m construction. Indeed, even olfactory receptors would be useless in many analytical applications, because the many chemicals described as ‘odorless’ would, by and large, fail to stimulate a man-made array built exclusively from olfactory receptors.

Accordingly, to realize the utility of the olfactory paradigm in designing and using chemical biosensors and biodiscriminators analytically, there is a need for engineered receptors that can be assembled into arrays or ensembles of receptors wherein the receptors of a given array have overlapping specificities for a family of ligands, and wherein the overlap (which ultimately determines a given array's specific utility) can itself be engineered so as to confer upon the array as a whole the ability to discrimate among chemically distinct occupants of a defined region of chemical space.

Disclosed herein is a method of making engineered GPCRs for use in such a biodiscriminator. Others, for example, Lemer (U.S. Pat. No. 6,475,733), Alberte (U.S. Pat. No. 6,692,696), Ault, et al. (Abstracts, 1^{st} Int'l Mtng. Synthetic Biol., p. 8, 2004) and U.S. Pat. Publ. 2005/0074834 have made mutant GPCRs by random mutagenesis. It has been proposed to create receptors capable of detecting “non-natural” ligands such as TNT in this manner. The cited references also mention site-directed mutagenesis, without elaboration. In each reference, the olfactory paradigm was duly noted, but the objective of creating receptors for use in arrays to discriminate among related compounds was not pursued, and n>m construction was not contemplated. What is needed is a method of making elements for such arrays, wherein the array is adapted to discriminate among ligands. Such a method is provided herein. In one embodiment, the invention teaches to first generate a highly sensitive (hypersensitive) mutant, preferably by random mutagenesis, followed, preferably, by site-directed mutagenesis of that mutant to form a family of mutants having overlapping specificities within a specified family of ligands.

There is, further, a need for a method or algorithm to construct arrays or ensembles of sensor elements from an inventory of such elements, which method will permit the artisan to construct a biodiscriminator adapted to the analysis of related chemicals in any region of chemical space that is of interest. An algorithm suitable for assembling an array of receptors responsive to a family of ligands that compete for occupancy on the receptors is provided. The method allows the practitioner, given knowledge of the relevant dissociation constants of receptors in inventory, to predict the performance of an array constructed from a selection of sensor elements. According to the method, the artisan can predict the performance of the array at various concentrations of two or more analytes dispersed in an environment (e.g., dissolved in a buffer solution) at various analyte compositions. The predictions are tested and critical deviations corrected by selecting a different sensor element where indicated. Because the tests of performance rely on routine techniques well-known in the art, optimization for a particular purpose is straightforward.

To build a chemical sensor ensemble in accordance with the invention, in particular a biodiscriminator that is capable of discriminating among a relatively large number of distinct ligands without requiring an equally large number of receptors, one first needs a supply of receptors from which to draw an array that is diverse in two senses: (i) the array should include receptors that are not monospecific, i.e., that have some degree of responsiveness to several members of the ligand family of interest, and (ii) the array should include receptors that collectively span the spectrum of the ligand family. The array should also have connectivity in the sense that overlapping sensitivities connect the array as a whole. Once a library of such elements is secured, a biodisciminator array can be assembled by drawing from the library a set of elements appropriate to the discrimination task at hand.

The present invention provides such elements by taking advantage of the tendency of GPCRs to be “promiscuous,” that is, not stringently mono-specific. The present invention, moreover, harnesses that tendency by generating mutants from a single parent, thus controlling overlap. Finally, to provide the diversity needed to span an entire ligand family with relatively few receptors, the present invention relies on a particular mutation strategy to generate the GPCRs. The strategy first finds a mutant that is highly sensitive to a representative ligand of the ligand family of interest, and then utilizes knowledge of the structural biology of the particular GPCR to derive from that parent several mutants to occupy diverse regions of the array. These mutants may be sensitive to analytes that induce no response whatever from the original, naturally occurring or wild-type GPCR.

Because the invention can thus dramatically increase the repertoire of chemical compounds detectable with a single discriminator construct, the discriminator can serve as a highly efficient platform for screening collections of chemical compounds, particularly structurally and sterically related compounds.

Biological assays are frequently utilized for chemical detection because of their convenience, as well as their high degree of sensitivity and specificity relative to alternative means of chemical analysis. Presently, the vast majority of biological assays for chemical analytes are based on monoclonal antibodies or coupled enzyme assays. The methods and molecules provided herein open new regions of chemical space to biochemical analysis by utilizing a different class of chemical receptor, G protein-coupled receptors (GPCRs), for analysis. The addressable region of chemical space includes all GPCR ligands, which encompass nearly 40% of drug compounds currently on the market, and chemically related compounds, which would likely include the synthetic precursors of many drug compounds. The invention relies on repeated mutagenesis and selection of receptors to increase the breadth of the chemical repertoire recognizable by GPCRs, from which one can create ensembles of receptors capable of discriminating among related chemicals that could not be distinguished by a single naturally-occurring receptor. The ease of mutagenesis and selection for this chemical analysis system is far greater than for coupled enzyme assays, and the ensemble of chemicals that could be analyzed by GPCRs encompasses an economically relevant set of compounds, many of which are not readily addressable by alternative techniques.

The detection methods and molecules provided are also distinct from those used in alternative chemical detection technologies in that the GPCRs used for chemical analysis are linkable to cellular signal transduction pathways in a variety of eukaryotic cells. This affords the opportunity to design genetically selectable chemical screens, in which the chemical analyte of interest is capable of controlling the life or death, or other properties, of a cell. Currently, neither enzymatic assays nor antibody-based chemical assays can be effectively linked to cellular signaling pathways in a systematic way.

Here, a yeast system developed for functional expression of heterologous GPCRs was used as a platform to create novel receptors. That is, a gene for a “foreign” GPCR (in this case, a mutated human GPCR) is made to express itself in yeast cells in such a way that activation of the foreign GPCR now affects the behavior of the yeast cell. Yeast strains that have been utilized for functional analysis of G protein-coupled receptors and drug screening were constructed by taking advantage of similarities between the yeast mating response pathway and human signal transduction pathways (Silverman, L., et al. (1998) Curr Opin Chem Biol 2(3):397-403). In yeast, the α and a mating pheromones are ligands for the Ste2 and Ste3 GPCRs, which signal through a heterotrimeric G protein and a MAP kinase pathway to regulate physiological and transcriptional outputs of the mating response (Marsh, L., et al. (1991) Annu Rev Cell Biol 7:699-728). By replacing the yeast pheromone receptor with a mammalian GPCR, tailoring the G-protein to couple the mammalian GPCR to the pheromone response pathway and engineering the output of the pheromone response pathway, strains have been generated whose growth depends on functional activation of the inserted mammalian receptor. Such strains have allowed genetic selection to identify receptor ligands, genetic analysis of ligand structure, and genetic selection of constitutively active receptors (Manfredi, J. P., et al. (1996) Mol Cell Biol 16(9):4700-9; Klein, C., et al. (1998) Nat Biotechnol 16(13):1334-7; Zhang, W. B., et al. (2002) J Biol Chem 277(27):24515-21; Arias, D. A., et al. (2003) J Biol Chem 278(38):36513-21; Sachpatzidis, A., et al. (2003) J Biol Chem 278(2):896-907; Celic, A., et al. (2004) Methods Mol Biol 237:105-20). Using this system, the present invention also provides methods for identifying receptors with novel ligand recognition properties.

To initiate the study, isolated mutants of the human UDP-glucose receptor (KIAA001, P2Y14) with altered ligand specificity were sought. The UDP-glucose (UDPG) receptor is part of a large family of nucleotide receptors, some of which have affinity to sugar nucleotides (Abbracchio, M. P., et al. (2003) Trends Pharmacol Sci 24(2):52-5). Sugar nucleotides are key reagents in the biological or chemo-enzymatic synthesis of carbohydrates. Sugar nucleotides are structurally diverse, with similar physicochemical properties, thus posing a challenging target for inexpensive, high-throughput chemical analysis. Accordingly, sugar nucleotide sensors like the human UDPG receptor were a good starting point for the development of chemosensors that can be used to assay sugar nucleotides and their derivatives.

The present invention provides in one of its aspects a method of creating a family of UDPG receptors. Random mutagenesis of the entire receptor gene, followed by genetic selection for growth in the presence of ligand, was used to identify receptors sensitized to all the ligands that normally interact (to one degree or another) with the wild-type receptor, with essentially unaltered ligand preference. Then, by targeting mutagenesis to motifs in the receptor anticipated by the inventors to interact with ligand, receptor mutants different in both ligand specificity (but not necessarily different in kind) and efficacy were created.

Among the receptors generated by targeted mutagenesis were a receptor with ‘inverted’ stereochemical preference for UDP-Galactose (UDP-Gal) versus UDPG, and a receptor that is more robustly activated by a partial agonist, UDP.

As one example of how engineered receptors can be utilized in a combinatorial manner, pairwise application of engineered receptors was used to uniquely identify an unknown ligand with a single pair of measurements. This demonstrates the feasibility of a combinatorial approach to detector design using engineered receptors. Possible applications would include air and groundwater monitoring, biohazard detection, and drug testing.

The method for creating new GPCR-based molecules preferably involves the successive mutagenesis and selection of GPCRs based on sensitivity to chemical ligands. By repeatedly mutagenizing and selecting for novel ligand binding properties, it is possible to create a panel (inventory, library) of chemically sensitive receptors with overlapping ligand-recognition properties that would collectively function in a manner analogous to the human olfactory system. The steps required for isolating receptor mutants, and the properties needed for application to chemical sensing, are detailed below.

### EXAMPLE 1

Materials. UDPG, UDP-galactose (UDP-Gal), UDP-N-acetylglucosamine (UDP-glcNAC), UDP-N-acetylgaltactosamine (UDP-galNAC), uridine triphosphate (UTP), uridine diphosphate (UDP), glucose-1-phosphate (G-1-P), glucose-6-phosphate (G-6-P), UDP-glucose-pyrophosphorylase (UGPase), glycogen synthase (GS) pyrophosphatase (PPase) and fluorescein (FDG) were purchased from Sigma-Aldrich (St. Louis, Mo.). Mutazyme® was purchased from Stratagene.

Strains and plasmids. Mutagenesis and selection were performed in yeast strain CY10560 (P_{FUS1}-HIS3 ade2Δ3447 ade8 Δ3457 can1-100 far1Δ442 his3Δ200 leu2-3,112 lys2 sst2Δ1056 ste14::trp1::LYS2 ste18γ6-3841 ste3Δ1156 trp1-1 ura3-52). β-Galactosidase assays were performed using yeast strain CY10981 (P_{FUS1}-HIS3 can1-100 far1Δ1442 his3Δ200 leu2-3,112 lys2 sst2Δ2 ste14::trp1::LYS2 ste3Δ1156 trp1-1 ura3-52) carrying plasmid Cp1021 (P_{FUS1}-LacZ 2 μm URA3). The UDP-glucose receptor was cloned into plasmid Cp1651 to yield plasmid pAH1 (P_{PGK1}-hP2Y14 2 μm LEU2) for expression in the host strains.

Mutagenesis and selection of sensitized receptor mutants. The entire UDPG receptor gene was mutagenized via error-prone mutagenesis (see, for example, U.S. Pat. No. 6,803,216) to an estimated frequency of ˜2-5 mutations/kb following the Mutazyme® protocol. A library of mutants was generated by gap repair cloning (see, for example, Ma, H. et al., (1987) Gene 58:201-16). A population of each such mutant was grown (“plated”) to near confluence on selective media. 1-2×10⁵ colonies were screened by replica plating to SC-His media (Kaiser, C., et al. (1994) Methods in yeast genetics : a Cold Spring Harbor Laboratory course manual. Cold Spring Harbor, N.Y., Cold Spring Harbor Laboratory Press) with and without ligand. Yeast growth media were supplemented by 1 mM 3AT, a competitive inhibitor of the HIS3 reporter gene product, which sets the threshold for reporter gene activation. The reporter gene, in this context, simply provided a means of confirming that yeast cells intended to be recipients of the GPCR of interest did in fact accept the GPCR. Yeast cells that have incorporated a foreign gene are referred to as “transformants.”

Targeted mutagenesis and selection of functional receptor mutants. To generate targeted mutants, oligonucleotides with randomized sequences, corresponding to the codons to be mutagenized, were utilized to generate overlapping PCR products. The method and many applicable variations thereof are known in the art. See for example, U.S. Pat. Nos. 6,448,048 and 6,878,531, both of which are incorporated herein by reference. The HIAR motif corresponds to P2Y14 amino acids 250-253 in TM6, the KExT motif corresponds to amino acids 277-280 in TM7, the NMY motif corresponds to amino acids 104-106 in TM3, and the AxxFY motif corresponds to amino acids 98-102 in TM3. Mutant libraries were generated by gap repair using overlapping PCR products and transforming to media selective for recombined plasmids. To select for functional mutants, libraries were replica plated (that is, plates having surfaces that cannot support growth of certain organisms in a population are “infected” by pressing a “master” plate against their surfaces, which master plate is supporting various strains in the population) to selective media containing one of six ligands: UDP-Gal, UDPG, UDP-galNAc, UDP-glcNAc, UDP or dTDP-glucose (50 μl 1 mM spread on 30 ml SC-Leu-His agar medium in 8.5 cm petri plates).

β-galactosidase assays. β-Galactosidase assays were carried out as described previously (Chambers, J. K., et al. (2000) J Biol Chem 275(15):10767-71), with the exception that cultures were incubated with ligand in 500 μl cultures in 48-well culture blocks rather than in microtiter plates as described. Schild plot analysis was carried out as described, using visual interpolation to read inhibitor concentrations corresponding to the EC₂₀ of each plot (Limbird, L. E. (1996) Cell surface receptors: a short course on theory and methods. Boston, Kluwer Academic Publisher).

Genetic selection of sensitized receptor mutants. Studies were initiated to redirect the ligand specificity of the human UDPG receptor by random mutagenesis of the complete gene, followed by selection for mutants responsive to non-native ligands. Yeast strain CY10560 expressing the wild type human UDPG receptor gene grows on selective medium with 0.3 μM UDPG (8.5 cm petri plates spread with 100 μl of 10⁻⁴ M UDPG over 30 ml solid medium) but does not grow on plates with one-tenth that concentration of UDPG. In addition, the strain fails to grow on selective medium containing 0.3 μM UDP-Gal, UDP-glcNAc or UDP-galNAc, so these ligands are considered to be non-native ligands (Chambers, J. K., et al. (2000) J Biol Chem 275(15): 10767-71); that is, the wild-type receptor is not naturally adapted to bind these chemicals.

Cells were transformed with a plasmid library carrying a randomly mutated human UDPG receptor gene. Transformants were recovered on non-selective medium and then screened for growth on selective media without ligand, or containing 0.03 μM UDPG, or 0.3 μM UDP-Gal, UDP-glcNAc or UDP-galNAc. Some of the transformants exhibited constitutive growth in the absence of ligand. Most of the nonconstitutive mutant receptors that promoted growth in response to any one of the non-native ligands showed growth in response to each of the other ligands, including UDPG.

The sensitized receptors facilitated subsequent receptor engineering experiments. Receptors were subjected to sequential rounds of mutagenesis to determine if they could exhibit even greater sensitivity, while potentially accumulating more substantial changes in ligand specificity. Plasmid DNA was recovered from those transformants that exhibited ligand-dependent growth with enhanced sensitivity to non-native ligands, the DNA samples were pooled, and further mutagenesis and selection procedures were performed. DNA was then extracted from several candidate clones exhibiting enhanced, ligand-dependent growth. An additional round of mutation and selection was performed on each individually. This cycle was repeated using the best candidate clones from the third round.

After these four rounds of mutagenesis and selection, the preponderance of non-constitutive mutants exhibited enhanced response to all four ligands. Using plate-based growth assays, in which patches of sensitized mutants were replica plated to media supplemented with varying concentrations of ligand, it was not possible to discern changes in the relative sensitivity to ligand for any of the sensitized mutants. However, in growth assays the most sensitive mutant receptors responded to approximately thirty-fold lower concentrations of UDPG than did wild type receptor. The apparent sensitivity did not change significantly from the third to the fourth cycle of mutagenesis and selection.

Several UDPG receptor mutants were selected for sequencing. Mutations were scattered across the receptor gene, suggesting that few, if any, of the effects of mutations were caused by changes to residues that interact directly with ligand (Table 1).

Isolation of specificity mutants via targeted mutagenesis. Since random mutagenesis of the UDPG receptor appeared to yield a preponderance of mutants with increased sensitivity but unaltered specificity, mutagenesis focused on residues that were hypothesized to be directly involved in ligand binding was undertaken. As a starting receptor for this directed mutagenesis, a receptor designated 2211 that was isolated as described above (Table 1) was selected.

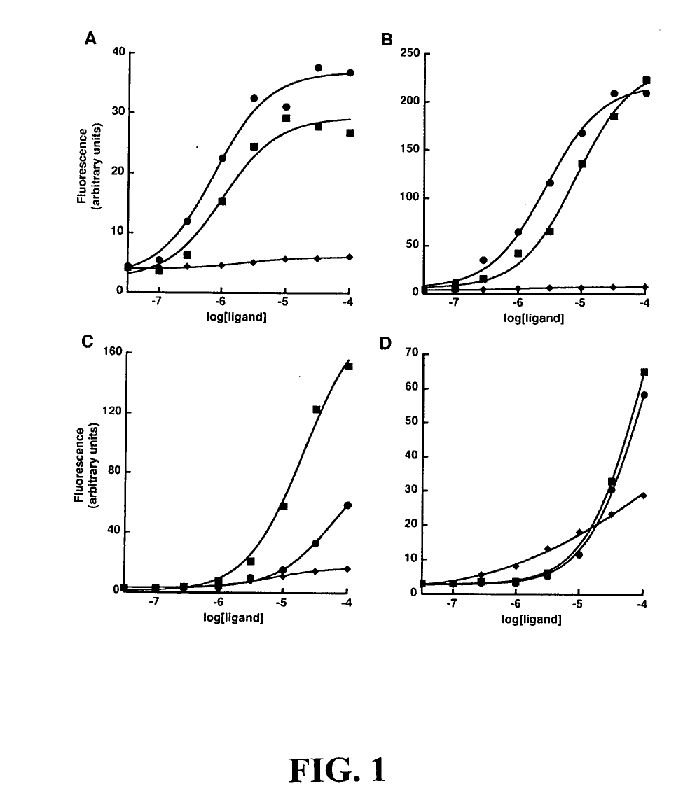

The mutant 2211 receptor responds to all three non-native ligands tested, and to significantly lower concentrations of UDPG than the wild type receptor in growth assays. In liquid β-galactosidase assays, as described in materials and methods, this receptor shows increased reporter activation at all concentrations of ligand tested, without significant changes in the EC₅₀ (FIG. 1).

Similar to the wild type UDPG receptor, the 2211 receptor did not promote detectable growth in plate assays in the absence of ligand. The 2211 receptor retained the essential signaling properties of the wild type P2Y14 receptor, while functioning more robustly in the yeast expression system, it thus constituted a better starting point for subsequent rounds of mutagenesis and selection.

Motifs were selected to target for mutagenesis based on structural data, in particular, conserved residues in the nucleotide receptor subfamily and a model of the transmembrane regions of the UDPG receptor based on the crystal structure of bovine rhodopsin (Moro, S., et al. (1998) J Med Chem 41(9):1456-66; Palczewski, K., et al. (2000) Science 289(5480):739-45; Jacobson, K. A., et al. (2004) Curr Top Med Chem 4(8):805-19). Overall, alignments of the transmembrane domains and conserved residues suggested to the inventors a ligand binding pocket in the canonical ligand-binding region of GPCRs between transmembrane helices 3, 6 and 7.

The ‘HIAR’ motif was first targeted in transmembrane domain 6. The His250 and Arg253 residues in P2Y14 correspond to His and Lys residues, respectively, that are critical for activation of the P2Y1 receptor by ATP (Moro, S., et al. (1998) J Med Chem 41(9):1456-66; Jacobson, K. A., et al. (2004) Curr Top Med Chem 4(8):805-19). One of the mutations, A252V, in the sensitized 2211 mutant falls in this motif, although it is not yet known if this specific mutation gives rise to a sensitized phenotype.

Libraries containing the randomized HIAR motif were constructed in vivo by cotransforming strain CY10560 cells with three DNA fragments: a 2211 receptor plasmid cut to remove the HIAR domain, and a pair of PCR products synthesized with oligonucleotides randomized over the HIAR region and with 5′ and 3′ extensions overlapping both sides of the gap in the plasmid.

Transformants were replicated to plates containing UDPG or one of the non-native ligands UDP-gal, UDP-glcNAc or UDP-galNAc. Transformants were also replica plated onto plates containing UDP and dTDP-glucose to test for the presence of mutant receptors capable of responding to ligands that do not activate the parent receptor. 20 out of ˜5000 transformants grew in the presence of one or more ligands.

The twenty receptors isolated in this primary screen were subsequently retested for growth in the presence of lower concentrations of each ligand, to ascertain whether the receptor had significant changes in ligand preference. In this secondary screen only one of the twenty receptors had a dramatically different profile of ligand responsiveness than the starting receptor.

To determine whether the receptors that lacked appreciable changes in ligand specificity were indeed mutants, and to verify the complexity of the mutant library, the DNA encoding each receptor was sequenced. Sequencing revealed that 3 plasmids contained unaltered 2211 receptor DNA. Thirteen of the 17 remaining plasmids contained unique, readable sequences, each of which contained randomized DNA across the HIAR motif. Strikingly, the histidine residue was conserved in every mutant receptor and the arginine residue was conserved in 12 of the 13 clones (Table 2). The remaining clone contained HTVK in place of the HIAR motif and was the only mutant that exhibited altered ligand specificity in growth assays, showing a preference for growth in the presence of UDP-Gal versus UDPG.

The HTVK mutant, designated H-20, was selected as the template for mutagenesis of three additional motifs, ‘AxxFY’ and ‘NMY’ in TM3, and ‘KExT’ in TM7. These mutants were tested in the same manner as the HIAR mutants, focusing only on mutants with clear changes in relative growth on one or more ligands. Of these, one mutant, designated K-3, in which KEFT was replaced by KGFT, had the most dramatic changes. The K-3 mutant grew poorly relative to its parent in response to UDPG and UDP-Gal, but surprisingly grew in the presence of UDP.

Thus, targeted mutagenesis of conserved motifs in ligand binding domains of the UDPG receptor can yield receptors with altered ligand specificity, as has been shown in developing the H-20 and K-3 mutants as detailed above.

Quantitative analysis of ligand binding to mutant receptors. To further analyze the properties of the mutant receptors with altered ligand specificity, GPCR activation in vivo was quantified as a function of ligand type and concentration (FIG. 1). The reporter assays confirmed qualitative observations from plate assays indicating a relative order of ligand activation of UDPG>UDP-Gal>>UDP for 2211; UDP-Gal>UDPG>UDP for H-20; and UDP >UDPG≈UDP-Gal for K-3.

The H-20 receptor has lower EC₅₀'s for both UDPG and UDP-Gal than does the 2211 receptor. The lower values preclude determination of maximal activation levels for either ligand against H-20 and, accordingly, precise measurement of EC₅₀ values. Also, one cannot determine from these data whether UDPG is a partial or full agonist for H-20, although UDPG shows no competitive antagonist activity toward UDP-Gal activation of H-20 (data not shown). Similarly, analysis of the K-3 receptor was complicated by the inability to fully activate the receptor with the available ligands. However, given the comparatively strong activation of the receptor at high concentrations of UDP-Gal and UDPG, UDP likely acts as at least a partial agonist for this receptor.

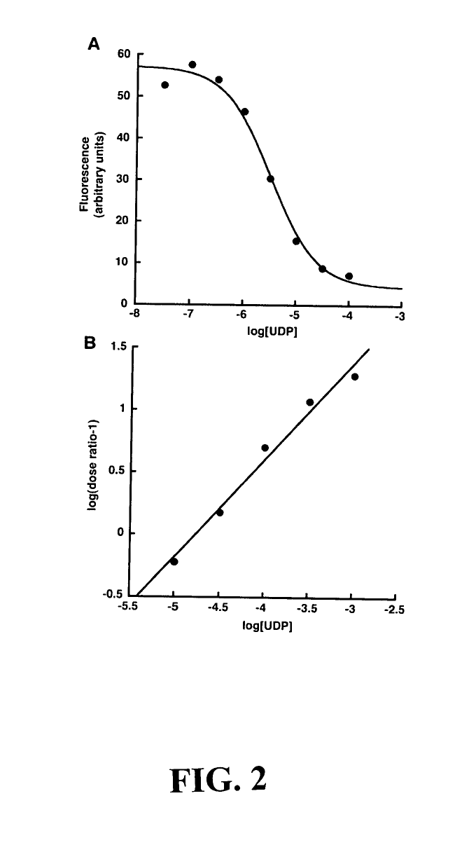

Initially, the observation that UDP activates both the H-20 receptor and the K-3 receptor suggested that the mutant receptors had gained an affinity for UDP. Rather, careful examination showed that the 2211 receptor is weakly activated by UDP. Quantitative analysis revealed that UDP acts as a competitive inhibitor of UDPG activation of 221 1 (FIG. 2a). A comprehensive analysis of the inhibition characteristics of UDP as a function of different agonist concentrations revealed an apparent K_{D} of ˜10^{-4.5} M of UDP for 2211 (FIG. 2b). This suggests that the effect of the H-20 and K-3 mutations is to change the consequence of UDP binding to the receptor (from antagonism to partial agonism), as opposed to generating a new site for UDP binding to the receptor.

Engineered GPCRs as chemical sensors. The isolation of receptors with distinct, but overlapping, specificities toward different ligands allowed us to explore novel uses of GPCRs as chemical sensors. Receptors were tested to determine if they could function in a combinatorial fashion, such that a small number of receptors could be used to uniquely identify multiple compounds. With a single receptor, it is for the most part impossible to differentiate among pure solutions of different receptor ligands. Even with extensive controls, it would be impossible to differentiate between a dilute solution of a strong agonist and a concentrated solution of a weak agonist. In contrast, using multiple receptors with overlapping ligand recognition properties, it should be possible to establish for each ligand a signature “written” in the combinatorial data forthcoming from a receptor array. These “signatures” would then differentiate one ligand from another.

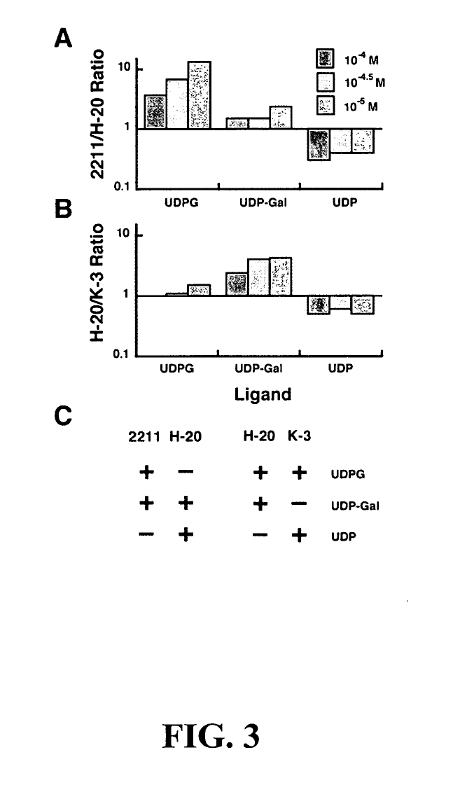

This can be illustrated in an intuitive way by calculating the ratio of responses for a pair of receptors, using the data underlying FIG. 1. For each receptor pair, the ratio of reporter activity at each ligand concentration was calculated (Table 3). Focusing on the H-20/K-3 ratios (FIG. 3a), it can be seen that at concentrations of ligand above 10⁻⁵ M, UDP-Gal stimulation resulted in a ratio greater than 2.4, stimulation with UDPG resulted in a ratio between 1.0 and 1.5 and stimulation with UDP resulted in a value less than 0.6. Thus, given an unknown solution containing UDP, UDP-Gal, or UDPG, a single determination of the ratio of activity of the two receptors across this range of concentrations would uniquely identify the compound in the solution. A similar result holds for the H-20/221 1 receptor pair (FIG. 3b), although not for the 2211/K-3 pair.

Thus, a single measurement from only two receptors allows precise discrimination of three different analytes. Further, discrimination is achieved over more than a ten-fold range in concentration of analyte and is independent of absolute response of either receptor. This is laid out most intuitively in FIG. 3c, which shows pictorially how the identity of each ligand can be expressed simply in terms of the relative response of the two receptors, and how application of a third receptor introduces a redundant criterion for discrimination. The response of the mutant receptors also distinguishes these three analytes from virtually all other analytes, since the three compounds each activate the mutant receptors while virtually all other analytes do not.

Although the results are less reliable at lower ligand concentrations, given a sample of unknown concentration it would be possible to carry out a simple set of controls to ascertain if the sample is in the appropriate range of concentrations.

Biodiscriminator GPCR elements. Using a yeast system for functional expression of G-protein coupled receptors, standard yeast genetic and culture techniques were applied to create and isolate receptors with altered ligand recognition properties. The sequential application of mutagenesis and selection has not previously been applied in this way to GPCRs, in part due to a lack of a facile genetic system in which to conduct such studies. Through application of such sequential mutagenesis and selection, mutants of the UDPG receptor with one or more of enhanced sensitivity, changes in ligand specificity, and changes in efficacy, were isolated. The resulting receptors have properties that are amenable to chemical sensing applications.

The initial efforts to obtain mutant receptors with altered ligand specificity by random mutagenesis of the entire UDPG receptor gene yielded mutants with increased sensitivity in response to ligands but none with changes in ligand specificity. The EC₅₀s of the mutant receptors with increased sensitivity are similar to or greater than the EC₅₀ of the wild type UDP-glucose receptor in yeast. This suggests that the increased sensitivity of this set of receptor mutants does not result from an enhanced affinity of the receptor for ligands but more likely arises from either an increased concentration of functional receptor numbers in the cell or from an increased specific activity of mutant receptors (i.e. an increased ability of ligand bound receptor molecules to activate the associated G-protein).

The fact that mutations yielding receptor activation are scattered across the receptor gene suggests that a number of positions in the primary structure of the protein can affect either the efficiency of its biosynthesis, through changes affecting steps in the trafficking or maturation of the receptor, or its specific activity. This large number of sites whose mutation results in activation may account for predominance of activated receptors relative to those with altered ligand preference following random mutagenesis of the entire gene.

From a protein engineering standpoint, generation of sensitized receptors is akin to generation of functionally optimized enzymes. Many functional parameters of catalytically useful enzymes have been optimized, including thermal stability and specific activity, without the goal of altering enzymatic substrate specificity (Turner, N. J. (2003) Trends Biotechnol 21(11):474-8). Like functionally optimized enzymes, the sensitized UDPG receptors are in and of themselves, useful tools.

Subsequent screens for specificity mutants were simplified by the robust responses to nonnative ligands by the 2211 receptor. The use of yeast strains expressing the 2211 receptor as whole-cell ‘indicator’ assays for extremely low concentrations of sugar-nucleotides secreted by growing cells is also contemplated herein.

UDPG receptor mutants with altered ligand specificity were created by targeting regions of the molecule likely to be involved in ligand interaction, on the basis of homology and structural modeling. The observed phenotypic effects of mutagenesis fit standard pharmacological models for receptor function. Mutants in the ligand binding pocket would be expected to alter either the relative affinity of the receptor for different ligands, the consequences of a ligand's binding to the receptor or both.

The transformation of UDP from a weak partial agonist to a stronger partial agonist of the K-3 receptor is preferably an example of a change in the consequence of ligand binding, i.e. a change in efficacy. Pharmacological evaluation of the affinity of UDP leads to the conclusion that all of the receptors have similar affinities for UDP. In the H-20 and K-3 receptors, UDP functions as an increasingly strong partial agonist, whereas in the cases of the wild type and 2211 receptors the ligand functions primarily as an antagonist.

The relative affinity of the K-3 receptor for the UDPG and UDP-Gal ligands appears to be diminished, based on the dramatically higher EC₅₀'s of the two compounds. In contrast, the H-20 receptor appears to have reduced affinity for UDPG, but similar affinity for UDP-Gal relative to that of the parent 2211 receptor. Thus, in this case, the effect of the mutation has been to diminish the interaction of one, but not another, ligand for the receptor. To date, no mutant receptor with increased affinity for a ligand has been recovered. This may be attributable in part to the fact that UDP, one of the two compounds utilized in screening not expected to bind to the UDPG receptor, was in fact a ligand, while the other compound, dTDP-glucose, differs from the UDP-sugars in the base and the deoxy ribose, moieties distal to the phosphates and UDP-sugars.

Parallels can be constructed between engineering ligand specificity of receptors and engineering substrate recognition by biocatalytic enzymes. Directed evolution is frequently utilized to fine tune the chiral specificity of a biocatalytic transformation, typically with the goal of creating or enhancing a bias in substrate recognition to obtain an optically pure product (May, O., et al. (2000) Nat Biotechnol 18(3):317-20; Reetz, M. T. (2004) Methods Enzymol 388:238-56). In such cases mutants may be selected for substrate specificity at the expense of achieving maximal turnover (May, O., et al. (2000) Nat Biotechnol 18(3):317-20).

This situation is analogous to the experience of redirecting ligand specificity at the expense of maximal receptor sensitivity to any one ligand. This would be counterproductive to the common engineering goal of generating maximal stereospecificity for a biocatalyst. Here, substantially changing preference for one stereoisomer versus another, regardless of the exact ratio of affinities, was the goal.

In fact, the H-20 mutant has ‘inverted’ chiral specificity vis a vis the 2211 receptor, as opposed to an enhancement or refinement of the 2211 preference for UDPG versus UDP-Gal. Receptors with a high level of discrimination, on the order of the >100:1 ratio typically sought for biocatalysts, may be unnecessary or even disadvantageous for some chemical sensing applications, as olfactory sensors have been postulated to function more robustly if the individual receptors are relatively broadly tuned (Alkasab, T. K., et al. (2002) Chem Senses 27(3):261-75).

Finally, while the processes of engineering receptor and enzyme specificity may be conceptually analogous, there are important distinctions at the mechanistic level. Interactions between enzyme and substrate are typically transient and involve binding affinities of substrates, products, and transition states. The chemical motifs subject to stereochemical discrimination could be in the catalytic center, or far from it. In contrast, interactions between receptors and their ligands, which are not chemically altered by binding, can be more kinetically stable, while the efficacy of each ligand may vary. Thus it would be inappropriate to overstate the similarities of engineering receptor specificity versus enzymatic substrate specificity.

GPCRs as biosensors. The recovery of receptors with distinct but overlapping ligand recognition properties has allowed the exploration of aspects of chemoreception presumed to underlie olfaction. Applying chemical receptors as sensors in a combinatorial manner would create a powerful new tool for chemical detection. Even without engineering, GPCRs are remarkable chemical sensors, and any GPCR can be utilized as a chemical sensor when expressed in cells that allow ligand binding to be coupled to an easily measured output, the many expression systems developed for GPCR drug screening being cases in point.

Since many GPCR ligands are drugs, the universe of chemical compounds addressable by GPCR biosensors is scientifically and economically relevant. In some cases such receptor-based assay systems have intrinsic advantages over other systems for chemical sensing, like enzyme-linked colorimetric assays. For instance, it may be difficult to link chemical ligands to colorimetric assays, or it may not be feasible to purify the ligands in question from a mixture that confounds chemoenzymatic detection. In these experiments the ligands are in fact in complex mixtures for example with yeast growth media and yeast cells. They are thus are presented in a complicated mixture, typical of a biological HTS scenario, that would defeat numerous alternative means of chemical detection.

The instant invention provides mutant receptors (and method for generating them) that dramatically extend the power of receptors as chemical sensors, simply by adjusting the relative sensitivity of the receptors to certain ligands so that the receptors can be utilized in a combinatorial manner. This principle has been exemplified by highlighting conditions in which ‘pure’ samples of receptor ligands can be unambiguously identified over a ten-fold range in concentrations. This example was chosen for simplicity and clarity, but the principle is a powerful one and this mode of chemical analysis can certainly be extended with additional receptors in miniaturized assays.

Biological molecules have a history of use as sensitive, effective biosensors. Enzyme assays coupled to colorimetric outputs are standard tools for chemical detection, while monoclonal antibodies are ubiquitous tools for detection of biomolecules. Biological chemical receptors as a class have been underutilized as chemical sensors, due in part to the perception that cell-based assay systems are valuable primarily as drug screening technologies rather than as chemical sensing technologies. The instant invention provides in its various aspects engineered GCPRs with altered ligand interactions, as well as combinatorial application of engineered GPCRs—which clearly offers potential for development of quick, inexpensive screens for stereo- or enantioselective biocatalytic transformations, or for trace amounts of bioactive agents. Such tools are a valuable resource for the scientific community. Methods for further creation and development of engineered GPCRs are also provided herein.

### EXAMPLE 2

Here we analyze a number of factors relating to the performance and limits of resolution for chemosensory arrays, with particular emphasis on the potential to screen for functional mutants of GPCRs. At an arbitrary point in chemical space the maximum discriminatory capacity of an array will be defined by only two receptors in the array. To provide a quantitative framework for our discussion of chemosensory resolution we present a model for the idealized interactions of two analytes with two generic chemical receptors. We also describe how nonlinear stimulation of receptor response would be expected to change array performance. Finally, we analyze how receptors might be designed or selected to achieve maximal discriminatory potential in the context of an array, while simultaneously achieving breadth of coverage.

For our purposes any receptor can function as a chemical sensor element, provided its output signal is roughly hyperbolic as a function of chemical ligand concentration (Signal ∝1/(1+k/[S]). For certain tasks only one chemical sensor element needs to be employed, as long as the element can faithfully respond to changes in the concentration of the analyte of interest. For instance, if a receptor assay is employed in an enzyme-engineering project where the goal is to improve the specific activity of an enzyme toward a certain substrate, the amount of product will be proportional to the specific activity of the enzyme, and sensors that are responsive to the product can be used to screen for enzymes that produce the most product.

The one-reaction-one-sensor scenario is appealing in its simplicity, and engineering chemical receptors promises to dramatically extend the range of enzymatic transformations that can be assayed in such a facile manner. However, many biocatalytic reactions call for more sophisticated screening tools. For instance, when the goal is to refine the activity of an enzyme so that it more selectively produces one of a variety of possible products, a single chemosensory readout often will not be sufficient. This is because product mixtures must be screened for their relative, as opposed to their absolute, concentration. For example, when the goal is to refine the activity of an enzyme so that it favors the production of one stereoisomer over another, a single sensor cannot differentiate candidates that have improved stereospecificity from those with altered specific activity (FIG. 1). Some, if not most, desirable candidates will have lower overall activity but improved selectivity. Thus some chemosensory applications call for the use of arrays of chemical sensors in order to allow mixtures of products to be characterized. This leads to the question of how receptors can be utilized to resolve small differences in chemical composition of mixtures.

For a given pair of chemical analytes, the resolution of a chemical sensor refers to the uncertainty in a measurement of the composition and concentration of a mixture of the two analytes. The amount of uncertainty in a given measurement will itself vary as the composition and concentration of the mixture changes. Accordingly, chemical resolution might best be described in terms of the uncertainty of each measurement as a function of the composition and concentration of a chemical mixture.

For simplicity, consider a scenario in which two receptors detect two ligands with no significant crosstalk. In this case two sensors are responsible for chemical detection that have little overlap in ligand binding (i.e. assuming there is little cross-activation and the two ligands do not compete for receptor occupancy) but each receptor responds sensitively to its ligand. Further assuming for the moment that the signal of each receptor is proportional to its occupancy, the ratio of the signals from each chemical sensor can be expressed as being proportional to the occupancy of the ligand binding site. If the receptors in this example were enzymes their signals would be proportional to the Michaelis constants for the enzymes. For other types of receptors the signal is presumed to be proportional to the Kd between ligand and receptor. In each case, the signal, plotted on a semi-log plot, appears to be a sigmoidal curve as a function of ligand concentration.

Each ligand activates to the extent that it is bound by the receptor, proportional to the efficacy of the ligand. A straightforward derivation of hyperbolic binding curves for a chemical ligand/substrate and competitive inhibitors can be found here. In the absence of spare receptors, the equation for ligand A signaling through a receptor is a hyperbolic binding curve.

$${R}_{1} effA \frac{1}{1 + \frac{{K}_{d A 1}}{[ A ]}} = {R}_{1} effA \frac{[ A ]}{[ A ] + {K}_{d A 1}} . \qquad Eq . 1$$

Where RᵢeffX is the efficacy of ligand X for receptor i, K_{d Xi} is the K_{d} for ligand X and receptor i and [X] is the concentration of ligand X.

Expressing the composition of a given mixture of ligands in terms of the mole fraction of each ligand and the total concentration of the mixture, the predicted ratios of receptor activation can be plotted (FIG. 2). This plot reveals a surface in which the gradient corresponds to the local change in responsiveness to changes in the composition and concentration.

In conjunction with this expression for the ratio of receptor activation, the error for each measurement needs to be taken into account. If the error is 10% for each measurement, for instance, the uncertainty in the measurements can be projected onto the gradient representing the intrinsic resolution of the system. Because the intrinsic resolution varies as the composition and concentration of the mixture varies, even with consistent errors in measurements there will be differences in the overall resolution of the system. Additionally, one might expect increases in measurement errors at progressively lower concentrations.

Effects of Partial Agonists and Inhibitors on the Model.

Standard pharmacological models allow us to further describe the effects of different forms of cross-activation on the resolving power of a chemical sensor, using estimates of the K_{d} and the efficacy of the individual ligands. Again, we utilized pharmacological models for receptor/ligand interactions under idealized conditions. We assumed that no spare receptors would be present, that there are no cooperative interactions between receptors or ligands, and that each ligand would be a competitive inhibitor of the others. Our model anticipates that the maximum output of the system could vary for different ligands; that is, ligands can have different efficacies. These assumptions appeared to be an appropriate starting point for modeling signaling by the UDP-glucose receptor and mutants expressed in the yeast system. Moreover, these assumptions would be expected to apply to many receptors interacting with competing ligands.

When two ligands compete for binding a single receptor, each ligand would be expected to act as a competitive inhibitor of the other. The equation for A binding in the presence of inhibitor B incorporates an additional term.

$${R}_{1} eff A \frac{1}{1 + \frac{{K}_{d A 1} ( 1 + \frac{[ B ]}{{K}_{d B 1}} )}{[ A ]}} = {R}_{1} eff A \frac{[ A ]}{[ A ] + {K}_{d A 1} ( 1 + \frac{[ B ]}{{K}_{d B 1}} )} . \qquad Eq . 2$$

This expression represents the contribution of A to receptor 1 signaling, and a second term can incorporate the contribution of ligand B to receptor 1 signaling.

$${R}_{1} eff A \frac{1}{1 + \frac{{K}_{d A 1} ( 1 + \frac{[ B ]}{{K}_{d B 1}} )}{[ A ]}} + {R}_{1} eff B \frac{1}{1 + \frac{{K}_{d B 1} ( 1 + \frac{[ A ]}{{K}_{d A 1}} )}{[ B ]}} . \qquad Eq . 3$$

This equation represents the cross-inhibition of each ligand by the other and the contribution of each ligand to receptor activation. Once again, this model does not take into consideration the potential effects of spare receptors. Rather, it is assumed that the contribution of each ligand to signaling is directly proportional to its receptor occupancy. Eq. 3 represents all possible combinations of A and B, but a given mixture subject to analysis would have a fixed ratio of concentrations to be measured at various concentrations in the course of analysis. Thus it is helpful to rewrite Eq 3 in terms of the two unknowns for a given mixture: the ratio of concentrations, and the total concentration of the mixture.

$${R}_{1} eff A \frac{1}{1 + \frac{{K}_{d A 1} ( 1 + \frac{( 1 - x ) [ Mix ]}{{K}_{d B 1}} )}{x [ Mix ]}} + {R}_{1} eff B \frac{1}{1 + \frac{{K}_{d B 1} ( 1 + \frac{x [ Mix ]}{{K}_{d A 1}} )}{( 1 - x ) [ Mix ]}} . \qquad Eq . 4$$

Here x is the mol fraction of ligand A, 1-x is the mol fraction B and [Mix] is the molar concentration of the two ligands combined. Eq. 4 can be used to graph the expected responses of Receptor 1 from different mixtures of ligands A and B over a range of concentrations simply by providing measured values for the Kd's and efficacy of each ligand. The same calculations can be performed for a second receptor, Receptor 2, and the ratio of responses can be calculated by taking the ratio of the predicted responses from Receptors 1 and 2. (FIG. 3.)

Once again it is possible to visualize the change in the ratio of responses as a function of the composition of a mixture of ligands. Taking into account the efficacy of each ligand implicly includes scenarios in which one ligand is an antagonist, as well as partial agonism.

Effects of Nonlinear Contributions to Receptor Signaling.

Feedback and cooperativity are important sources of nonlinear responsiveness for biological sensory systems. Another important factor in predicting and understanding the behavior of biological receptors as chemical sensors is the fact that the output signal is often saturable. For instance, transcription can be fully activated long before cell surface receptors are fully occupied. The previous scenarios assume a linear correspondence between receptor occupancy by the chemical ligand and signal, but only a few receptors on the cell surface may need to be active to induce maximal transcriptional response. Occupancy of the remaining receptors does not further induce signaling, and the unoccupied receptors are referred to as spare receptors. Many other scenarios can contribute to a nonlinear response between membrane receptor occupancy and strength of output signal, including cooperative ligand binding interactions, receptor desensitization, or other feedback loops that weaken or strengthen response to ligand binding. To illustrate the importance of these nonlinear contributions to signaling we will briefly consider the case of spare receptors.

The effect of spare receptors can be can be approximated by substituting the EC50 of a ligand for the Kd when calculating the contribution of that ligand to signaling, but more rigorous models have been developed previously. Even when spare receptors are present, inhibition by competing ligands is still proportional to receptor occupancy and can be modeled using the Kd of each ligand.

Our expression for sensor resolution helps clarify the relationships among factors that influence the performance of sensory arrays, including binding affinity, measurement error and nonlinear responses. Thus we are led to ask more specific questions about how cells integrate signals from different receptors to select developmental fate, or the limits of resolution in detecting chemical gradients during development.

The expression for sensor resolution serves as a means to anticipate the performance of a given set of receptors in a given system. Rather than relying exclusively on trial and error to establish the robustness of a certain assay, it becomes possible to predict the likelihood that a given set of sensors will be satisfactory for a given task. Understanding the interplay between ligand binding and sensor performance makes it possible to identify specific engineering goals required to achieve a desired standard of performance. Receptor properties can be engineered to a certain standard, or receptors can be selected from an ensemble of mutants so as to maximize the discriminatory power of an array over the set of chemicals that needs to be resolved. By maximizing the response gradient between each analyte pair it becomes possible to create an algorithm for sensor design that will optimize a sensory array for real world performance. In instances in which receptor affinity can be computationally refined it becomes possible to utilize an expression for sensor resolving power as a guideline for in silico evolutionary goals.

The rate at which new metabolic pathways can be developed for chemical production is limited partially by the rates at which enzymes can be identified, optimized and assembled into a given biosynthetic pathway. The unbending reality is that nearly every enzymatic step in a biosynthetic pathway will require, or at least benefit from, some level of engineering to create a more productive and cost-effective route to synthesis by fermentation. Two constraints limit the rate at which optimization of a biocatalytic transformation can be performed. The first constraint could be described as ‘design efficiency’, which refers to the rate at which successful or desirable candidate enzymes can be generated, typically in the context of a library of candidate designs. The second constraint is screening throughput, which refers to the rate at which designs can be tested and desirable candidates isolated. Tools for chemical screening are highly variable, and must be adapted to the very specific chemical environment of the target analytes.

It is the screening scenarios that have the fewest resources that represent the most important targets for improved tools. Tremendous accomplishments have been made in protein engineering when sufficiently powerful screening tools have become available.

For instance, phage display libraries allow antibodies to be selected from extremely large libraries. On the other end of the spectrum, some enzymatic transformations are currently essentially impossible to assay in the context of a cell or confounding chemical backgrounds. Among the most challenging category of screens are chemical screens in which multiple analytes must be monitored and compared. Numerous technologies have been proposed for constructing miniaturized sensory arrays, including the use of dyes, aptamers, whole cells or enzymes. Developing quantitative algorithms to assemble arrays for specific screening tasks will serve to speed chemical assay development.

### EXAMPLE 3

An algorithm to aid in the design of chemical sensors wherein the collective response of a set of receptors is expressed as a function of the composition and concentration of an ensemble of ligands, the receptor signals are proportional to ligand occupancy, and the interactions of receptors and ligands are competitive. The ligand ensemble takes the form of a dissolved mixture of ligands.

$$signal = {k}_{eff {R}_{i} {L}_{1}} ( \frac{1}{1 + \frac{{K}_{d {R}_{i} {L}_{1}}}{[ {L}_{1} ]} ( 1 + \frac{[ {L}_{2} ]}{{K}_{d {R}_{i} {L}_{2}}} + \frac{[ {L}_{3} ]}{{K}_{d {R}_{i} {L}_{3}}} … \frac{[ {L}_{j} ]}{{K}_{d {R}_{i} {L}_{j}}} )} ) + {k}_{eff {R}_{i} {L}_{2}} ( \frac{1}{1 + \frac{{K}_{{dR}_{i} {L}_{2}}}{[ {L}_{2} ]} ( \frac{[ {L}_{1} ]}{{K}_{d {R}_{i} {L}_{1}}} + \frac{[ {L}_{3} ]}{{K}_{d {R}_{i} {L}_{3}}} … \frac{[ {L}_{j} ]}{{K}_{d {R}_{i} {L}_{j}}} )} )$$

In this example Rᵢ is a given receptor and Lⱼ is a given ligand. ‘Signal’ is the signal for one receptor, given an ensemble of ligands. Signal is calculated for each Rᵢ in the array, and the collective responsiveness of the array is analyzed as a function of ligand composition of the ensemble.

Using this expression as a descriptor of sensory array output, it becomes possible to model the performance of a sensory array and to implement algorithms to optimize the design of sensory arrays. Essentially, one applies the following recursive design algorithm:

1. Minimize the number of receptors in the array. A panel of receptors and receptor mutants (>100) is applied to analyze an ensemble of 10 ligands. The objective is to create an array with as few receptors as possible that will maximize the ability to discriminate among the various ligands. For any ensemble of ligands, the resolution of the array is determined by the pair of receptors with maximal change in response to an incremental change in composition of the analyte ensemble. Out of a large panel of receptors, some receptors are expected not to contribute to enhancing the resolution of the array, regardless of the composition of the analyte ensemble. These are ‘redundant’ receptors. By considering all combinations of ligands over a range of ligand concentrations, and modeling the responses of each combination of receptors, the differential signal from the receptors is maximized and redundant receptors are eliminated.

2. Quantify the incremental contributions of the remaining receptors. The set of receptors yielded by the above computations are the receptors required to achieve maximal resolution, but some receptors are similar to other receptors in the array. The incremental change in sensor resolution caused by deleting a receptor from the array is calculated to quantify that receptor's contribution to array resolution. Receptors contributing below a predetermined threshold are deleted. Receptors above the threshold but having similar effects vis a vis determining the limits of sensor resolution are identified. This step is akin to ‘clustering’ receptors. From each highly related ‘cluster’ at least one member is included in the final array.

3. Quantify the weaknesses of the array in terms of the ability to resolve certain combinations of ligands. Once the optimal set of receptors is determined, the resolution as a function of ligand composition is analyzed to identify strengths and weaknesses in array design.

a) Identify regions of poor resolution in ligand space. Array outputs over several regions of ligand space are compared, and regions in which there is poor resolution (that is, where the output of the array tends not to vary with the species of ligands sought to be distinguished from one another) are identified.

b) After a set of receptors having the requisite resolution is acquired, array output is observed over several combinations of the ligand species sought to be distinguished from one another to identify any regions of ligand space where one or more ligands masks a signal of the array.

c) Array output is further evaluated over a range of ligand combinations to identify metamerisms, i.e., regions of ligand space in which different ligand combinations produce identical patterns of receptor activation at certain concentrations.

d) Identify ligands that are uniquely identified over all proposed combinations of ligands.

4. Create new receptor design objectives based on the properties of the receptor array that is ‘in hand’. Design of receptor-ligand interactions is inherently governed by discrete changes in molecular structure of the receptor molecule(s). In many cases the trajectory through chemical space is also constrained by, say, the genetic code or the chemical nature of the assay system. Similarly, the rate at which new receptors can be generated and tested can impose a time constraint on receptor design. Even with hypothetical computational resources that could predict binding constants of receptor candidates, it is unlikely that the precise properties of novel receptors can be anticipated before the receptors are designed.

These physical limitations conspire to ensure that molecular design is not deterministic. Even if one has a clear picture of the optimal properties of a putative array, there is no guarantee that the requisite receptors will be designable, evolvable, or synthesizable. Thus design goals must fluctuate along with the emergent behavior of the chemical system that is being designed.

Our algorithm addresses this key principle by making it possible to anticipate the strengths and weaknesses of various hypothetical arrays. Having an understanding of sensor activation in the presence of ensembles of ligands will be an invaluable tool in planning and executing subsequent receptor design and selection.

Tables

TABLE 1
Receptor mutants.

| Mutant | Parent | Mutations | Positions |
|---|---|---|---|
| 1 | wt | W128C | IL2 |
| 5 | wt | Y137C, S237I | 4.41, EL2 |
| 2-1 | Pooled | G80S, Y137C, S237I | 2.63, 4.41, EL2, |
| | round 1 | | |
| 2-10 | Pooled | W128R, L148F, S237C, | IL2, 4.52, EL2, IL3 |
| | round 1 | L314I | |
| 2-2 | Pooled | K54E, A193V, A252V | IL1, 5.45, 6.54 |
| | round 1 | | |
| 2-2-1 | 2-2 | K54E, A193V, F243I, | IL1, 5.45, 6.45, 6.54 |
| | | A252V | |
| 2-2-1-1 | 2-2-1 | K54E, A98V, A193V, | IL1, 3.29, 5.45, 6.45, |
| | | F244I, A252V | 6.54 |
| H-20 | 2-2-1-1 | 2-2-1-1 + 250-253 | 6.52-6.55 |
| | | HIAR−>HTVK | |
| K-3 | H-20 | H-20 + E278G | 7.36 |

A subset of UDP-glucose receptor mutants were isolated as described and analyzed by sequencing. Numbered mutants were isolated by screening libraries generated by gene-wide random mutagenesis for sensitization to receptor ligands. The second round of screening utilized a pool of mutants isolated in the first round of mutagenesis as template. Lettered (H-20, K-3) mutants were generated by targeted saturation mutagenesis of the indicated motifs. Positions are indicated as extracellular loop 1-3 (EL1-3), intracellular loop 1-3 (IL1-3) or transmembrane domain (numbered according to Ballesteros {Ballesteros, 1995 #60}).

TABLE 2

Sequences of recovered HIAR mutants.

| | | |
|---|---|---|
| Clone | AA position | DNA sequence |

| | | | | | |
|---|---|---|---|---|---|
| | H | I | A | R | |
| H-1 | H | A | V | R | CAC GCG GTG AAG |
| H-2 | H | A | L | R | CAC GCA TTG CGG |
| H-5 | H | A | T | R | CAC GCG ACA AGA |
| H-9 | H | A | T | R | CAT GCG ACC CGG |
| H-17 | H | A | T | R | CAT GCC ACT AGA |
| H-6 | H | V | L | R | CAC GCG TTG CGT |
| H-7 | H | I | C | R | CAT ATT TGC CGG |
| H-8 | H | T | L | R | CAC ACG CTG CGA |
| H-10 | H | V | I | R | CAC GTT ATC CGA |
| H-11 | H | T | V | R | CAT GTG ACA AGG |
| H-13 | H | L | T | R | CAC TTG ACG CGT |
| H-14 | H | L | T | R | CAT TTA ACA AGG |
| H-20 | H | T | V | K | CAT ACC GTC AAG |
| recovered | H | AVILT | VILTC | RK | |
| aa's | | | | | |

13 unique mutant sequences were obtained from a set of receptors that displayed responsiveness to ligand. Mutant H-20 has a phenotype with substantial changes in ligand specificity. The remaining mutants recognize the same set of ligands, with the same relative ligand sensitivity as the parent 2211 receptor and the wild type P2Y14 receptor, although with varying degrees of overall receptor sensitivity. The positions corresponding to ‘I251’ and ‘A252’ tolerate substitution but exclude aromatic or charged residues.

TABLE 3
Ratio of receptor activation by UDPG, UDP-gal or UDP

| | 2211/H-20 | | | 2211/K-3 | | | H-20/K-3 | | |
|---|---|---|---|---|---|---|---|---|---|
| [ligand] | UDPG | UDPGal | UDP | UDPG | UDPGal | UDP | UDPG | UDPGal | UDP |
| −4.0 | 3.7 | 1.5 | 0.3 | 3.7 | 3.5 | 0.2 | 1.0 | 2.4 | 0.5 |
| −4.5 | 6.8 | 1.5 | 0.4 | 7.5 | 6.0 | 0.2 | 1.1 | 4.0 | 0.6 |
| −5.0 | 13.3 | 2.4 | 0.4 | 19.6 | 10.1 | 0.2 | 1.5 | 4.2 | 0.5 |
| −5.5 | 15.7 | 3.4 | 0.6 | b.d. | 18.9 | 0.3 | b.d. | 5.5 | 0.5 |

Receptor activation in response to the indicated concentration (expressed as the log₁₀ value) of each of three ligands (UDPG, UDPGal and UDP) was determined in vivo by reporter gene assays as described in materials and methods for each of the UDPG receptor subtypes (2211, H-20 and K-3). Presented are ratios of receptor activation for all three pairs of the three receptors for each ligand at each concentration. Values are not provided for those cases in which ligand activation of one or both of the receptors was less than two-fold background (b.d.=below detection).

What is claimed:

1. A plurality of sensor elements, wherein at least two of said sensor elements are capable of sensing
a) independently, at least two chemical species, and
b) in common, one of said at least two species.

2. The sensor elements of claim 1 wherein said sensing comprises binding of at least one of said species.

3. The sensor elements of claim 1 wherein said element comprises a biological molecule.

4. The sensor elements of claim 3 comprising a biodiscriminator.

5. The sensor elements of claim 3 wherein said biological molecule comprises a GPCR.

6. The sensor elements of claim 5 wherein said GPCR comprises a naturally occurring GPCR.

7. The sensor elements of claim 5 wherein said GPCR comprises a mutant GPCR.

8. The sensor elements of claim 7 wherein said mutant GPCR is hypersensitive.

9. The sensor elements of claim 7 wherein said mutant GPCR is made by random mutagenesis.

10. The sensor elements of claim 7 wherein said mutant GPCR is made by site-directed mutagenesis.

11. The sensor elements of claim 5 wherein said GPCR is functionally expressed in a living cell.

12. The sensor elements of claim 10 wherein said living cell is a yeast cell.

13. The sensor elements of claim 11 wherein said GPCR is coupled to a reporter system.

14. The sensor elements of claim 1 wherein said at least two species are isomers.

15. The sensor elements of claim 13 wherein said isomers are stereoisomers.

16. A method of making a chemical sensor comprising:
a. selecting a plurality of sensor elements of said sensor wherein at least two of said sensor elements are capable of sensing (i) independently, at least two chemical species, and (ii) in common, one of said at least two species; and
b. providing a common environment for said sensing.

17. The method of claim 16 wherein said sensing comprises binding of at least one of said species.

18. The method claim 16 wherein said sensing of said species occurs with unequal sensitivity.

19. The method of claim 16 wherein said sensor element comprises a biological molecule.

20. The method of claim 19 wherein said chemical sensor comprises a biodiscriminator.

21. The method of claim 19 wherein said biological molecule comprises a GPCR.

22. The method of claim 21 wherein said GPCR comprises a naturally occurring GPCR.

23. The method of claim 21 wherein said GPCR comprises a mutant GPCR.

24. The method of claim 23 wherein said mutant is hypersensitive.

25. The method of claim 23 wherein said mutant is made by random mutagenesis.

26. The method of claim 23 wherein said mutant is made by site-directed mutagenesis.

27. The method of claim 21 wherein said GPCR is functionally expressed in a living cell.

28. The method of claim 27 wherein said living cell is a yeast cell.

29. The method of claim 28 wherein said GPCR is coupled to a reporter system.

30. The method of claim 16 wherein said at least two species of chemicals are isomers.

31. The method of claim 30 wherein said isomers are stereoisomers.

32. A method of optimizing an ensemble of chemical sensor elements comprising the steps of:
a) assigning the sensor elements of said ensemble to similarity clusters, and
b) excluding from each said similarity cluster all but one of said sensor elements.

33. A composition comprising an amino acid sequence at least 90% identical to SEQ ID NO:01, wherein said amino acid sequence comprises, at positions corresponding to positions 54, 98, 193 and 243, respectively, of SEQ ID:NO:01, a glutamic acid, a glycine, a valine and an isoleucine and, corresponding to position 252 of said SEQ ID NO:01, a V.

34. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, an alanine and a valine.

35. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, an alanine and a leucine.

36. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, a valine and a leucine.

37. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, an isoleucine and a cysteine.

38. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, a threonine and a leucine.

39. A composition comprising an amino acid sequence at least 90% identical to SEQ ID NO:01 (wild type), wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, a valine and an isoleucine.

40. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, a valine and a threonine.

41. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, a leucine and a threonine.

42. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251 and 252, respectively, of SEQ ID NO:01, an alanine and threonine.

43. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251, 252 and 253, respectively, of SEQ ID NO:01, a threonine, a valine and a lysine.

44. A composition comprising an amino acid sequence at least 90% identical to SEQ ID NO:01, wherein said amino acid sequence comprises, at positions corresponding to positions 54, 98, 193 and 243, respectively, of SEQ ID NO:01, a glutamic acid, a glycine, a valine and an isoleucine and, corresponding to positions 251, 252, 253 and 278, respectively, of SEQ ID NO:01, a threonine, a valine a lysine and a glycine.
